(19) United States
(12) Patent Application Publication
(10) Pub. No.: US 2009/0162430 A1
(43) Pub. Date: Jun. 25, 2009

(54) Gelatin Capsule With Extra Cap for Combined Therapies

(76) Inventors: Kariem Ezzat Mahmoud, Cairo (EG); Hossam El-Deen Fawzy Ahmed, Ismailia (EG); Salah El-Deen Moaz, Cairo (EG)

Correspondence Address: BUTZEL LONG;IP DOCKETING DEPT, 350 SOUTH MAIN STREET, SUITE 300, ANN ARBOR, MI, 48104, US

(21) Appl. No.: 12/085,492

(22) PCT Filed: Mar. 21, 2007
(86) PCT No.: PCT/EG2007/000010

§ 371 Date: May 23, 2008

(51) Int. Cl. A61K 9/48; A61P 43/00

(52) U.S. Cl. 424/453

(57) ABSTRACT

A new pharmaceutical dosage form for the administration of combined therapies without interaction between them is disclosed. It consists of an upper gelatin capsule, hard or soft, with extra lower hard gelatin cap. The upper capsule contains one or more biologically active agent and the lower hard gelatin cap contains other biologically active agent or agents. Other lower or upper caps could be added to said capsule containing other biologically active agents with the same principle.

### TECHNICAL FIELD

The invention relates to pharmaceutical dosage forms being a dosage form comprising different separated biologically active agents; mainly for oral administration.

### BACKGROUND OF THE INVENTION

Various types of pharmaceutical dosage form are known for oral dosing. Pharmaceutical capsules are well known, generally being intended for oral dosing. Such capsules generally comprise an envelope wall of a pharmaceutically acceptable, e.g. orally ingestible, polymer material such as gelatin, although other materials for capsule walls, e.g. starch and cellulose based polymers are also known. Such capsules generally have soft walls made by making a film on a capsule former, which is then allowed to dry. Rigid walled capsules made by injection moulding are also known, see for example U.S. Pat. No. 4,576,284, U.S. Pat. No. 4,591,475, U.S. Pat. No. 4,655,840, U.S. Pat. No. 4,738,724, U.S. Pat. No. 4,738,817 and U.S. Pat. No. 4,790,881 (all to Warner Lambert). These disclose specific constructions of capsules made of gelatin, starch and other polymers, and methods of making them by injection moulding of hydrophilic polymer-water mixtures. U.S. Pat. No. 4,576,284 specifically discloses such capsules provided with a cap which closes the capsule, and which is formed in situ on the filled capsule by moulding. U.S. Pat. No. 4,738,724 discloses a wide range of rigid capsule shapes and parts.

Multi-compartment capsules, including those of the type where each compartment has different drug release characteristics or for example contains a different drug substance or formulation are also known, for example in U.S. Pat. No. 4,738,724 (Warner-Lambert), U.S. Pat. No. 5,672,359 (University of Kentucky), U.S. Pat. No. 5,443,461 (Alza Corp.), WO 9516438 (Cortecs Ltd.), WO 9012567 (Helminthology Inst.), DE-A-3727894, BE 900950 (Warner Lambert), FR 2524311, NL 7610038 (Tapanhony NV), FR 28646 (Pluripharm), U.S. Pat. No. 3,228,789 (Glassman), U.S. Pat. No. 3,186,910 (Glassman), WO 01/08666 (SmithKline Beecham), WO 04/010978 (GlaxoSmithKline) among others. U.S. Pat. No. 4,738,817 discloses a multicompartment capsule with a similar construction to those of U.S. Pat. No. 3,228,789 and U.S. Pat. No. 3,186,910, made of a water-plasticised gelatin.

All the prior art dosage forms require the manufacture of special capsular parts which require special tooling and procedure for manufacture, filling and sealing; which severely limited the practical usage of such dosage forms on industrial scale. Also, some of them depended on attachment of two or more entire capsules or capsule like compartments of various sizes together with different conformations. This technique, beside being not easy in manufacture, gives a dosage form with multi air-filled head spaces which increases the size of the dosage form without being benefited from. Others like for example WO 99/30693 depended on inclusion of one capsule into another. This technique beside giving two head spaces, severely reduces the free volume of the outer capsule for drug incorporation because most of the volume is occupied by the inner capsule, leading to more enlargement of capsule size. Therefore there remains a need for a multi-compartment dosage form utilizing the available conventional capsular parts in an innovative way with mild modifications to facilitate industrialization and decrease costs and in the same time doesn't significantly increase the size of the dosage form for patient compliance.

### DETAILED DESCRIPTION OF THE INVENTION

It is an object of this invention to provide an alternative and improved pharmaceutical dosage form which provides simultaneous administration of two or more biologically active agents without physical or chemical interaction between them in order to simplify dosing regimen to increase patient compliance and enhance the stability of combined therapies.

Another object of this invention to utilize the available conventional capsular parts in an innovative way with mild modifications to facilitate industrialization and decrease costs and in the same time without significantly increasing the size of the dosage form for patient compliance.

Other objects and advantages of the invention will be apparent from the following description.

According to this invention a multi-compartment capsule is provided utilizing the conventional capsular parts and filling and sealing methods. It depends on the principle that the hard gelatin cap could fit in both ends of a hard gelatin body (base) of the same capsular size, as the body (base) possesses a uniform diameter. Also the hard gelatin cap can fit in an oblong soft gelatin capsule of a diameter comparable to the corresponding hard gelatin body diameter.

Depending on this principle and by simple modification of the filling process, the cap could be treated as a body and filled with other biologically active agent or agents, then it is made to fit into another filled capsule to give a multi-compartment capsule possessing two or more segregated biologically active agents.

The modified filling process include the following steps:
1. The upper capsule (FIG. 1., FIG. 2. and FIG. 3) whether hard or soft having said criteria is separately filled with one or more biologically active agent according to the known art.
2. The lower cap (FIG. 1., FIG. 2 and FIG. 3.) is treated as a body and filled with the other biologically active agent or agents. It is filled to the extent that leaves a space for the upper capsule to fit in to ensure good closure
3. The upper capsule is descended to fit in the lower cap.
4. The upper capsule and the lower cap are then sealed together. They are sealed via any conventional sealing procedure e.g.: fit-together encircling grooves, sealing fluid, ultrasonic sealing or banding. If the fit-together encircling grooves are used for sealing, therefore there is an encircling groove at the lower part of the upper capsule to fit with the other encircling groove at the lower cap

This modification is very simple and could be done industrially and with low costs. And this is an important advantage that the invention provides.

The dimensions of the upper capsule and the lower cap could be changed in accordance with formulation needs, so that the body of the upper capsule could be shortened and the lower cap could be lengthened so as to provide more space in the lower cap or for better sealing purposes.

Another advantage the invention is that it provides no extra head spaces which insures the most efficient use of the multi-compartment dosage form with minimum increase in the capsule size to enhance patient compliance.

The separation of different biologically active agents or their different forms by a physical barrier vastly enhances the stability of these agents within the dosage form. It insures no physical or chemical interaction which ensures delivery of different agents in their original, 100% efficacious form. This decreases the cost of the dosage form as no interaction studies will be done beside increasing its shelf life.

Capsules are versatile dosage forms and thus the upper and lower capsules could be filled with any form of a biological active agent e.g.: powder, granules, pellets, tablets, paste, semisolid, liquid and either instant or controlled release and the upper capsule itself could be sustained release or enteric coated. Thus the invention provides a tool for tailored delivery of each medication in accordance with patient needs.

More extra caps could be added either upper or lower, so that more biologically active agents could be incorporated. In this case the diameter of the extra cap could be changed to fit in the desired place according to formulation needs.

The current invention provides a cheap, efficient and easy to manufacture dosage form for combined therapies which is essential in the current medical trend. Combined therapies are essential in many world threatening diseases such as: HIV, TB and heart diseases, which are also more prominent in poor countries. Therefore there is an urgent need to supply combined therapies for such diseases efficiently without interaction, quickly without long period development of complex dosage forms and their complex equipment and cheaply utilizing available technology. In this context the current invention is an important step.

### BRIEF DESCRIPTION OF DRAWINGS

FIG. 1, wherein 1 is the upper hard gelatin capsule, 2 is the cap of the upper hard gelatin capsule, 3 is the body of the upper hard gelatin capsule and 4 is the extra lower cap.

FIG. 2, wherein 5 is the upper soft gelatin capsule and 6 is the extra lower cap.

FIG. 3, wherein 7 is the upper soft gelatin capsule, 8 is the extra lower cap and 9 is the extra upper cap.

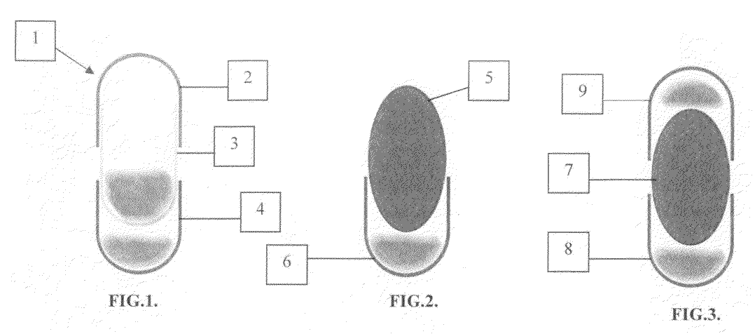

1-10. (canceled)

11. A pharmaceutical dosage form for the oral dosing of one or more biologically active agents, the dosage form comprising:
(a) a capsule having longitudinally opposite, hemispherical or hemi-ovoid first and second ends, the capsule defining a first sealed interior compartment containing a biologically active agent; and
(b) at least a first cap comprising a hard-shell capsule-half having a hemi-spherical or hemi-ovoid end, the capsule-half telescopingly received over one of the first or second ends of the capsule so that the hemi-spherical or hemi-ovoid end of the capsule is at least partially disposed within the first cap, the at least first cap being sealed relative to the capsule to define a second sealed interior compartment between the exterior of the capsule and the interior of the first capsule-half which is physically separated from the first interior compartment, and the second interior compartment containing a biologically active agent.

12. The pharmaceutical dosage form of claim 11, wherein the capsule is a soft-gel capsule.

13. The pharmaceutical dosage form of claim 12, wherein the biologically active agent in the first interior compartment and the biologically active agent in the second interior compartment are the same agent in the same pharmaceutical form.

14. The pharmaceutical dosage form of claim 12, wherein the biologically active agent in the first interior compartment and the biologically active agent in the second interior compartment are the same agent in pharmaceutically different forms.

15. The pharmaceutical dosage form of claim 12, wherein the biologically active agent in the first interior compartment and the biologically active agent in the second interior compartment are different agents.

16. The pharmaceutical dosage form of claim 12, further comprising a second cap comprising a hard-shell capsule-half having a hemi-spherical or hemi-ovoid end, the second capsule-half telescopingly received over the other of the first or second ends of the capsule so that the hemispherical or hemi-ovoid end of the capsule is at least partially disposed within the second cap, the second cap being sealed relative to the capsule to define a third sealed interior compartment between the exterior of the capsule and the interior of the second capsule-half which is physically separated from both the first and second interior compartments, and the third interior compartment containing a biologically active agent.

17. The pharmaceutical dosage form of claim 16, wherein two or more of the biologically active agent in the first interior compartment, the biologically active agent in the second interior compartment, and the biologically active agent in the third interior compartment are the same agent in the same pharmaceutical form.

18. The pharmaceutical dosage form of claim 16, wherein two or more of the biologically active agent in the first interior compartment, the biologically active agent in the second interior compartment, and the biologically active agent in the third interior compartment are the same agent in pharmaceutically different forms.

19. The pharmaceutical dosage form of claim 16, wherein two or more of the biologically active agent in the first interior compartment, the biologically active agent in the second interior compartment, and the biologically active agent in the third interior compartment are different agents.

20. The pharmaceutical dosage form of claim 11, wherein:
the capsule is a hard-shell capsule comprising a first capsule-half having a first outside diameter and one of the first and second hemi-spherical or hemi-ovoid ends, and a second capsule-half having the other one of the first and second hemi-spherical or hemi-ovoid ends and a second outside diameter which is greater than the first outside diameter, and wherein the second capsule-half is telescopingly received over the first outside diameter of the first capsule-half and sealed relative thereto to define the first sealed interior compartment; and
the at least first cap comprising having an outside diameter which is greater than the first outside diameter of the first capsule-half and being telescopingly received over the hemi-spherical or hemi-ovoid end of the first capsule-half.

21. The pharmaceutical dosage form of claim 20, wherein the at least first cap is characterized by an outside diameter that is substantially the same as the second outside diameter of the second capsule-half of the capsule, such that the second outside diameter defines the maximum outside diameter of the pharmaceutical dosage form.

22. The pharmaceutical dosage form of claim 20, wherein the biologically active agent in the first interior compartment and the biologically active agent in the second interior compartment are the same agent in the same pharmaceutical form.

23. The pharmaceutical dosage form of claim 20, wherein the biologically active agent in the first interior compartment and the biologically active agent in the second interior compartment are the same agent in pharmaceutically different forms.

24. The pharmaceutical dosage form of claim 20, wherein the biologically active agent in the first interior compartment and the biologically active agent in the second interior compartment are different agents.

25. A pharmaceutical dosage form for the oral dosing of one or more biologically active agents, the dosage form comprising:
(a) a soft-gel capsule having longitudinally opposite, hemi-spherical or hemi-ovoid ends, the capsule defining a first sealed interior compartment containing a biologically active agent; and
(b) at least a first cap comprising a hard-shell capsule-half telescopingly received over one of the ends of the capsule and sealed relative to the capsule to define a second sealed interior compartment between the exterior of the capsule and the interior of the first capsule-half which is physically separated from the first interior compartment, and the second interior compartment containing a biologically active agent.

26. The pharmaceutical dosage form of claim 25, further comprising a second cap comprising a hard-shell capsule-half telescopingly received over the other end of the soft-gel capsule and sealed relative to the capsule to define a third sealed interior compartment between the exterior of the capsule and the interior of the second capsule-half which is physically separated from both the first and second interior compartments, and the third interior compartment containing a biologically active agent.

27. A pharmaceutical dosage form for the oral dosing of one or more biologically active agents, the dosage form comprising:
(a) a hard-shell capsule comprising a first capsule-half having a first maximum outside diameter, and a second capsule-half having a second maximum outside diameter which is greater than the first maximum outside diameter, the second capsule-half being telescopingly received over the first capsule-half and sealed relative thereto to define a first sealed interior compartment containing a biologically active agent; and
(b) a first cap comprising a hard-shell capsule-half having an outside diameter which is greater than the first outside diameter of the first capsule-half and being telescopingly received over the first capsule-half and sealed relative thereto to define a second sealed interior compartment between the exterior of the first capsule-half and the first cap which is physically separated from the first interior compartment, and the second interior compartment containing a biologically active agent.
